(19) United States
(12) Patent Application Publication
(10) Pub. No.: US 2010/0152749 A1
(43) Pub. Date: Jun. 17, 2010

(54) Table-mounted surgical instrument stabilizers with single-handed or voice activated maneuverability

(76) Inventors: Walter von Pechmann, Bethesda, MD (US); Samuel C. Yoon, Clarksville, MD (US); Ben Lane, Phoenix, MD (US); Keith Lipford, Baltimore, MD (US); Austin Cox, Silver Spring, MD (US)

Correspondence Address: Ober, Kaler, Grimes & Shriver;Royal W. Craig, 120 East Baltimore Street, Baltimore, MD, 21202-1643, US

(21) Appl. No.: 12/584,366

(22) Filed: Sep. 3, 2009

Related U.S. Application Data
(63) Continuation-in-part of application No. PCT/US08/13661, filed on Dec. 12, 2008.

(51) Int. Cl. A61B 19/00

(52) U.S. Cl. 606/130

(57) ABSTRACT

A surgical instrument stabilizer system for securely holding virtually any instrument during any procedure, delivering vacuum when applicable, and yet allowing easy single-handed or voice activated repositioning by a user. The system utilizes opposing horizontal side-rails mounted to opposing sides of a surgical table, and opposing arms each slidably mounted on a side rail for global lengthwise positioning along the side rails. The arms carry a transverse beam at the foot of the operating table, and a flexible arm is movably mounted on the beam for global lengthwise positioning along the beam. Alternately, the flexible arm may be affixed directly to one of the existing horizontal side rails. The flexible arm leads to an instrument-supporting hand piece pivotally mounted for angular orientation of an instrument supported thereby. The flexible arm can be freely articulated to any position and locked in place. Thus, a supported instrument can be repositioned quickly and easily by single-handed or voice activated manipulation by a surgeon or surgical assistant, and locked in any position up and down along a vertical axis, forward and back, i.e, toward or away from the patient, and rotationally.

### CROSS-REFERENCE TO RELATED APPLICATIONS

The present application is a continuation-in-part of PCT Application No. PCT/U.S.08/13661, which derives priority from U.S. provisional patent application No. 61/005,746 filed 14 Dec. 2007.

### BACKGROUND OF THE INVENTION

1. Field of the Invention

The present invention relates to medical stabilizer methods and devices and, more particularly, devices that can be utilized to stabilize surgical instruments that would otherwise be hand held for maintaining exposure to a surgical site or otherwise.

2. Description of the Background

During surgery, whether open or laparoscopic, there is a need for a device that can be utilized to stabilize surgical instruments for various functions, most commonly for stabilizing instruments that would otherwise be handheld, in order to achieve desired tissue tension or to maintain exposure to the surgical site. Many such apparatuses already exist. For example, Cooper Surgical has recently released a hydraulically-controlled table mounted uterine manipulator stabilizer.

For example, United States Patent Application 20070129634 to Hickey et al. published Jun. 7, 2007 shows a biomedical positioning and stabilization system that allows the adjustable, yet rigid placement of a probe or other medical instrument against a region of interest/treatment on a patient. The system employs a flexible goose-neck armature attached to a rigid stand.

U.S. Pat. No. 5,170,790 to Lacoste et al. issued Dec. 15, 1992 shows a stabilizer system with a rigid arm hinged to a support stand at one end and to a ball-and-socket joint, a second arm reaching to an adjustable probe bracket.

U.S. Pat. No. 6,248,101 issued to Whitmore et al. (Barzell Whitmore Maroon Bells, Inc) on Jun. 19, 2001 shows an omni-directional precision instrument platform connected to stationary objects such as a portable floor stand and/or operating table.

U.S. Pat. No. 4,963,903 to Cane issued Oct. 16, 1990 shows a surgical camera positioning system that employs a flexible gooseneck.

U.S. Pat. No. 7,189,246 to Otsuka et al. (Olympus) issued Mar. 13, 2007 shows a medical instrument holding device with a tensor-lamp configuration leading to a ball-and-socket joint.

U.S. Pat. No. 6,514,239 to Shimmura et al. (Olympus) issued Feb. 4, 2003 shows a medical instrument holding apparatus likewise with a tensor-lamp configuration leading to a yoke-mounted support arm.

John R. Bookwalter has various patent for the various configurations and components of his retractor system, including U.S. Pat. Nos. 4,424,724 (expired); 4,254,763 (expired); 5,375,481 (active); and 5,520,608 (active). This system is made and marketed by Codman and employs a set of steel bars mounted to an operating table and used to suspend a steel ring above and around the surgical incision. The ring in turn serves as a docking site for instruments used to retract the wound edges.

There are variations within the Codman accessory line such as the Magrina vaginal retractor that can be used for similar surgical exposure during vaginal surgery. Other table mounted systems are available for similar functions, used to stabilize other devices such as the Martin's Arm (LTL Medical, LLC) that can be used to retain a camera in a fixed position during laparoscopy.

U.S. Pat. No. 6,958,038 to Feng et al. (Allegiance Corporation) issued Oct. 25, 2005 shows a multipositional ratchet-type stabilizer.

However, all of the foregoing systems require two free hands or one foot and one hand to change position of the stabilizing components, requiring the surgeon to basically put down whatever surgical instruments he or she may be using, adjusting the stabilizer and/or stabilized instruments, and then returning to the task at hand. This need for two hands or one foot and one hand to perform adjustments renders the existing devices inefficient and less amenable to frequent repositioning to suit the surgeon's needs. Additionally, the space required to use two hands for adjustments limits the application of these systems in situations where space is constrained, such as robotically assisted laparoscopic surgery.

As a specific example, during laparoscopic or robotically assisted sacral colpopexy, a vaginal probe is generally used to place the vagina on tension during dissection of anatomic spaces and subsequently during attachment of suspending graft to the vaginal muscularis. The probe is typically held in place by a surgical assistant because of the frequent need for repositioning of the probe to achieve the desired tissue tension. However, holding the probe in position is ergonomically awkward for an assistant standing at the bedside, particularly in the case of robotically assisted sacral colpopexy in which the surgical robot occupies the space between the patient's legs. The ideal solution would be to use a table mounted stabilizer that can hold the vaginal probe in the desired position, and yet allow repositioning of the vaginal probe quickly and easily enough to allow frequent repositioning by the surgeon or surgical assistant. For the sake of efficiency as well as to account for space constraints, the ideal table mounted stabilizer would be able to be repositioned with one hand (or voice activated).

A table-mounted stabilizer with these qualities would be useful for other operations that use hand held vaginal or rectal instruments such as uterine manipulators or vaginal retractors used during gynecologic surgery or rectal probes used during urologic and colorectal operations. Moreover, such systems could be used to stabilize the camera during laparoscopic surgery, or to maintain tissue positioning. Thus, it would be greatly advantageous to provide a device that can be utilized to stabilize surgical instruments for maintaining exposure to a surgical site or otherwise, which device can be adjusted and repositioned with one hand or by voice command.

### SUMMARY OF THE INVENTION

It is an object of the present invention to provide a table mounted stabilizer system capable of holding an instrument (camera, vaginal probe, etc.) or plurality of instruments in any desired position, which allows repositioning of the instrument(s) quickly and easily by single-handed or voice activated manipulation by a surgeon or surgical assistant, and which may also deliver a vacuum through the stabilized instrument to maintain tissue positioning.

It is another object to provide a table mounted stabilizer system as above that provides full adjustability of the location of the desired instrument up and down along a vertical axis, forward and back, i.e, toward or away from the patient, and rotationally. It is another object to provide a table mounted stabilizer system as above with a minimum load carrying capability of 2 ft-lbs torsionally, 25 pounds axially and 10 pounds laterally for rigid, reliable and secure support of any device.

It is still another object to provide a table mounted stabilizer system with the adjustability as above and yet which can be selectively locked into position for rigid, reliable and secure support of an instrument.

Other objects, features, and advantages of the present invention will become more apparent from the following detailed description of the preferred embodiments and certain modifications thereof. The stabilizer system serves to securely hold virtually any instrument during any procedure, for example, during laparoscopic sacral colpopexy which requires an absolutely stable probe during suturing of mesh to the vagina. The system utilizes currently available opposing horizontal side-rails mounted to opposing sides of a surgical table. A plurality of struts and adjustment mechanisms are then employed to create a plurality of separate adjustment points. Specifically, global lengthwise positioning is accomplished with a first component translationally attached to the side rails. Global unidirectional positioning is achieved with a flexible arm connected to an instrument-supporting hand piece. The instrument-supporting hand piece is pivotally mounted for angular orientation of the instrument supported thereby. In addition, some surgical tools that may be stabilized with this device require the use of vacuum through the tool and as such, this device provides for a vacuum feature. Thus, the present device allows easy single handed (or voice activated) repositioning in multiple directions (any direction, rotation or angle) when in an “unlocked” condition, a secure fixed position when in a “locked” condition, and with variable user-adjustable resistance there between, coupled with same hand (or voice activated)-control over the rigidity of the flexible arm combined with the delivery of vacuum when applicable.

### BRIEF DESCRIPTION OF THE DRAWINGS

Other objects, features, and advantages of the present invention will become more apparent from the following detailed description of the preferred embodiment and certain modifications thereof, in which

FIG. 1 is a side perspective view of an exemplary embodiment of the stabilizer system 1.

FIG. 2 is a side view of the stabilizer system 1 as in FIG. 1.

FIG. 3 is a composite front view of the stabilizer system 1 with enlarged insets.

FIG. 4 illustrates the presently-preferred flexible arm configuration used in the stabilizer system 1 of FIGS. 1-3.

FIG. 5 is an enlarged illustration of an exemplary flexible arm link 230 showing geometry.

FIG. 6 is an enlarged side-view of the flexible arm 20 supporting the hand piece 60, which is in turn carrying a vaginal probe 70.

FIG. 7 is a perspective view of an alternative configuration of the stabilizer system 1 in which the collar 50 and associated components are affixed directly to one of the existing horizontal side rails 12, without the use of the U-shaped frame 40.

### DETAILED DESCRIPTION OF THE PREFERRED EMBODIMENTS

As described above, the present invention is a system for stabilizing surgical instruments that would otherwise be handheld, or to maintain desired tissue tension or position or to maintain exposure to a surgical site or otherwise.

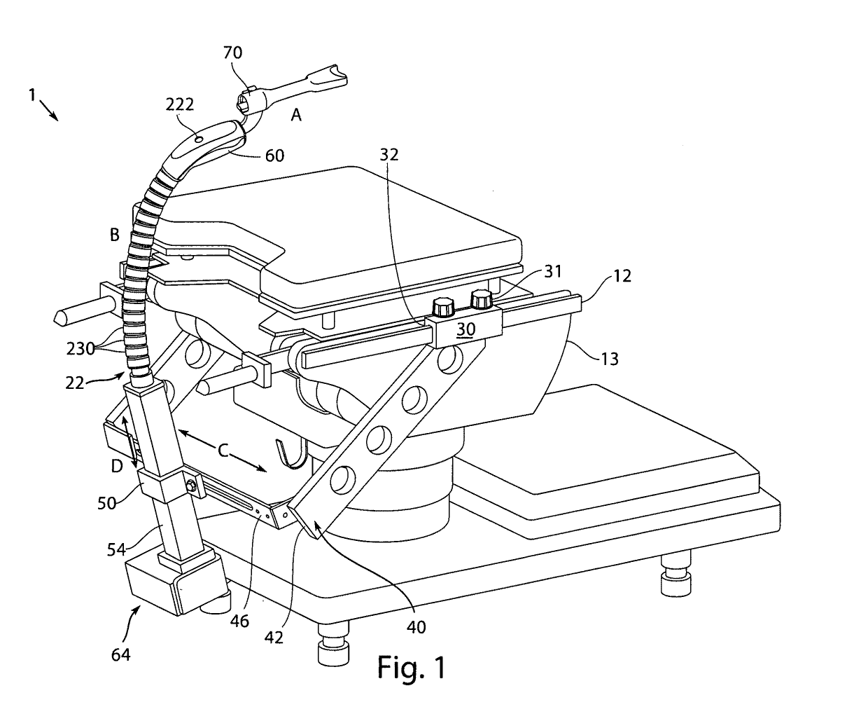

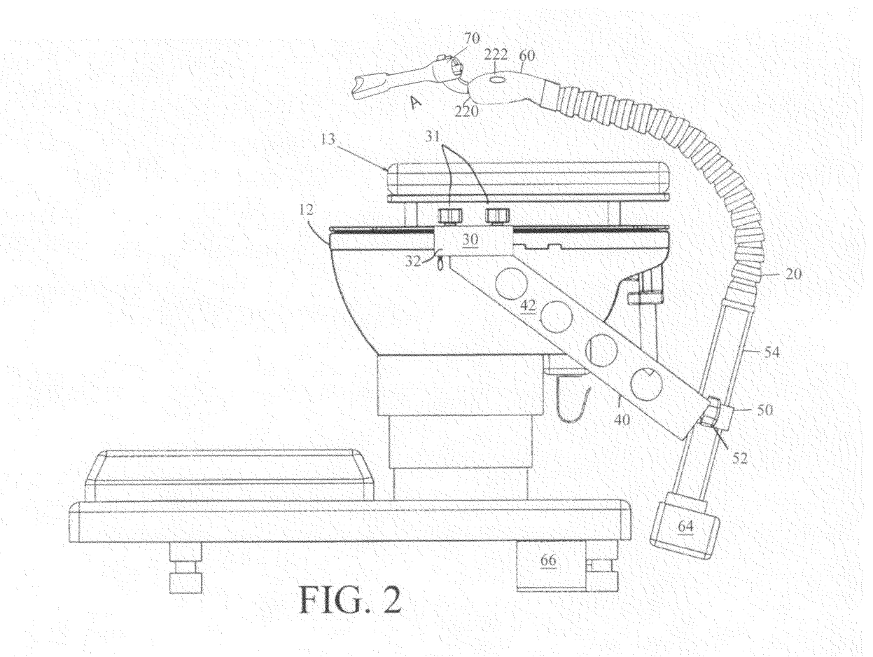

FIG. 1 is a side perspective view of an exemplary embodiment of the stabilizer system 1, and FIG. 2 is an opposing side view. The stabilizer system 1 utilizes the existing horizontal side-rails 12 mounted to the opposing sides of most surgical tables 13. The side-rails 12 are typically mounted on spacers so as to space them approximately 1″ outward from the table 13. Many surgical tables in the United States employ standardized North American side rails, which are well suited for present purposes. A lengthwise-translating quick-release bracket 30 is positioned on each side-rail 12 for tandem translation along one axis. Each quick-release bracket 30 further comprises a screw-clamp fixture having a clamp-jaw 32 extending around and behind the side-rail 12, and one or more knobs 31 for manually clamping the jaws 32 to the side-rails 12. The opposing quick release brackets 30 support a substantially U-shaped frame 40 that extends downward from one quick-release bracket 30 around the foot of the table 13 and to the other quick-release bracket 30 (obscured). U-shaped frame 40 further includes two (opposing) downwardly protruding angle arms 42 extending at an approximate 45 degree angle lengthwise toward the foot of the table 13 where they are joined to a transverse support arm 46. As illustrated, the angle arms 42 are preferably formed of extruded aluminum beams having spaced apertures extending along each face to maximize strength yet conserve weight. A collar 50 is adjustably mounted on the outer face of the support arm 46 for lateral sliding-translation there along. Collar 50 extends two pair of set screws into spaced tracks extending along the face of the support arm 46. The set screws are engaged into the tracks by a tongue-and-groove fit. Thus, while collar 50 cannot be detached it remains free to slide lengthwise. When desired, the set screws allow manual tightening of collar 50 against the support arm 46 to thereby prevent sliding, and thus a user can lock the collar 50 in any position along support arm 46 by tightening the set screws.

The collar 50 carries an articulating boom including an actuator 64 that extends an actuator housing 54 to a flexible arm 22 mounted there atop. The actuator housing 54 is adjustably mounted in the collar 50 and can be extended upward or retracted downward therein, and clamped in place. Specifically, the actuator 64 housing is slidably held within the yoke of the collar 50, and the yoke may be loosened if desired to extend or retract the actuator housing 54. A set screw with handle 52 (see FIG. 2) can be used to adjust and/or lock the actuator 64 in place.

An alternative scheme (described below) allows for fixation of the collar 50 and associated components described above directly to one of the existing horizontal side rails 12, without the use of the U-shaped frame 40.

The flexible arm 22 comprises a plurality of mating ball and socket links 230 optionally covered with a thin walled elastomeric sheath (not shown). The sheath is intended to prevent pinching of the patient or user between the flexible links 230, improves contamination control, improves sterilization capabilities and improves the aesthetics but otherwise performs no structural or operational function. A tensioning cable 240 (described below) runs throughout the arm 22 and actuator housing 54 and connects to actuator 64, the actuator 64 controlling the tension load on the cable 240. As the cable 240 tension is increased, the links 230 endure a corresponding increase in compressive load between each mating ball and socket. This loading system creates normal force loads between the ball and socket links 230, which in turn generates friction loads. The amount of friction is a function of a number of variables including the geometry of the links 230 (e.g., the size of the links 230), the materials of the links 230, the mating surface finishes, and the magnitude of the normal force on each link, all of which can be varied within the scope of the present invention. For any given design, the load between the links 230 (and the corresponding friction load) is controlled via the tension in the cable 240. As loads on each link are increased (with higher tension in the cable 240), each link imposes higher friction forces, which collectively alters the overall flexibility of the arm 22. Thus, at higher cable 240 tension, the arm 22 will endure external loads such as a surgeon pressing on a given tool that is being supported by the system. Therefore, greater tension in the cable 240 allows the arm 22 to support higher loads at the end of the arm 22 that supports a given tool, due to the increased friction between the links 230. At low cable 240 tension, there is little or no friction between the links 230 and the arm 22 can be manually moved and positioned by the operator with little or no resistance.

The movement of the cable 240 in the actuator 64 (and hence the tension in the cable 240) may be controlled in various ways. For example, the tension in the cable can be controlled by a button 222 mounted on an instrument-supporting hand piece 60, which button 222 may be connected to actuator 64 via a hard wired connection, optical, infrared, RF, Bluetooth (or other wireless communication). When used with a hard wired or optical connection, a slip ring or rotating connector is preferably incorporated in the hand piece 60 to allow the wires or optical fibers to slip relative to the mating link 230 and therefore prevent damage to the wires or fibers that could occur when the hand piece 60 is over rotated by the operator relative to the actuator 64. Alternatively, button 222 may be replaced with a foot pedal controller in communication with the actuator 64. Alternatively, button 222 may be replaced with a voice activated control system in communication with the actuator 64. A suitable voice activated control system includes a microphone coupled to an audio amplifier in turn coupled to a processor capable of running voice recognition software, such as Dragon Naturally Speaking™ software commercially available from Nuance™, or Fonix FAAST™ software commercially available from Fonix Corporation, 1225 Eagle Gate Tower, 60 East South Temple, Salt Lake City, Utah. The processor may be the actuator 64 control system. Button 222 (or foot pedal or voice control) provides for convenient same hand (or voice)-operated control over the amount of flex imparted to the arm 22. Pressing the button 222 (and holding it in this orientation), causes the cable 240 in the actuator 64 to extend or grow in length which reduces the amount of tension in the cable 240 by a predetermined amount and thereby reducing the friction load between the links 230 such that the flexible arm 22 can move in a more relaxed manner as chosen by the operator. Releasing the button 222 causes the cable 240 in the actuator 64 to retract in overall length which increases the amount of tension in the cable 240 by a predetermined amount, thereby increasing the friction load between the links 230 such that the flexible arm 22 has increased rigidity to a selectable degree chosen by the operator. At maximum tension the arm 22 becomes fully rigid in that the links 230 will not rotate relative to each other unless the maximum loading of the arm 22 is exceeded. In the case of voice activation, the operator will be able to control the movement and location of the flexible arm 22 through oral messages interpreted by an on-board processor such as the actuator 64 control system.

An ergonomic instrument-supporting hand piece 60 is mounted to the distal end of the flexible arm 22, and the hand piece 60 accepts a variety of probe-adapter inserts 220 each of which serves as a conforming receptacle for insertion of a probe. In the illustrated embodiment a vaginal probe 70 is mounted in the adapter 220 on the instrument-supporting hand piece 60 (although most any instrument or imaging device may be so mounted with a conforming probe-adapter insert 220).

The primary goals of the present device are to: 1) allow easy single handed repositioning of the arm 22 in multiple directions (any direction, rotation or angle) when in an unloaded or “unlocked” condition; 2) to provide a secure fixed position when in a “locked” condition; and 3) to provide the user with variable user-adjustable resistance there between, coupled with single-hand or voice activated-control over the rigidity of the flexible arm 22. This would be well-suited for situations requiring frequent repositioning, such as for stabilization of a laparoscopic camera, and would be equally suited for stabilizing a rectal probe, uterine manipulator, vaginal probe 70 or similar surgical tools. The need for adjustable resistance arises from the different types of instruments requiring stabilization. For instance, a table mounted stabilizer used to hold a laparoscopic camera in a fixed position requires only minimal resistance because it need only resist the weight of the camera itself. In contrast, a stabilizer used to hold a vaginal probe used during laparoscopic sacral colpopexy or a uterine manipulator during laparoscopic hysterectomy would require substantially greater resistance in order to keep the probe stable during suturing of mesh to the vagina. The present invention accomplishes this by including the ability to adjust the overall tension in the cable 240.

The invention provides multiple features to adjust the location and operating range of the flexible arm 22 via five separate adjustments settings A thru E as described below:

At point A, the instrument-supporting hand piece 60 is pivotally mounted to the distal end of the flexible arm 22 for angular orientation of the instrument supported thereby.

Point B is an adjustable-tension unidirectional adjustment point for local positioning implemented by the flexible arm 22 (controlled by button 222 or voice) with internal tensioning cable 240 (allowing multi-axis positioning by the plurality of ball and socket links 230).

Point C allows transverse global positioning of both the flexible arm 22 and instrument-supporting hand piece 60. This is accomplished by the collar 50 which is translatable lengthwise along support arm 46.

Point D allows global vertical positioning of the combined actuator 64, flexible arm 22 and instrument-supporting hand piece 60 by virtue of the actuator 64 which may be slidably repositioned within the collar 50

Finally, Point E allows global lengthwise positioning of both the flexible arm 22, instrument-supporting hand piece 60, actuator 64, support arm 46, collar 50 and U-shaped frame 40 along the surgical table by virtue of the quick-release bracket 30, which is movably mounted to the side-rail 12. The friction of the quick-release bracket 30 may be adjusted by knobs 31.

The foregoing configuration facilitates easy single-handed (or voice activated) local and global multi-directional repositioning of the supported instrument and variable resistance-setting. The range of motion is significantly increased. Surgical instruments can be supported in any location and any orientation within a 60 centimeter diameter spherical range of the point of origin of the instrument-supporting hand piece 60.

Moreover, selection of the material for the flexible elements 230 (high modulus of elasticity), combined with manufacturing requirements for controlled and uniform surface finish (injection molding) as well as the design of the optimum geometry of the flexible elements 230 has enabled the load carrying capacity of the foregoing configuration to be substantially greater than prior art stabilizers.

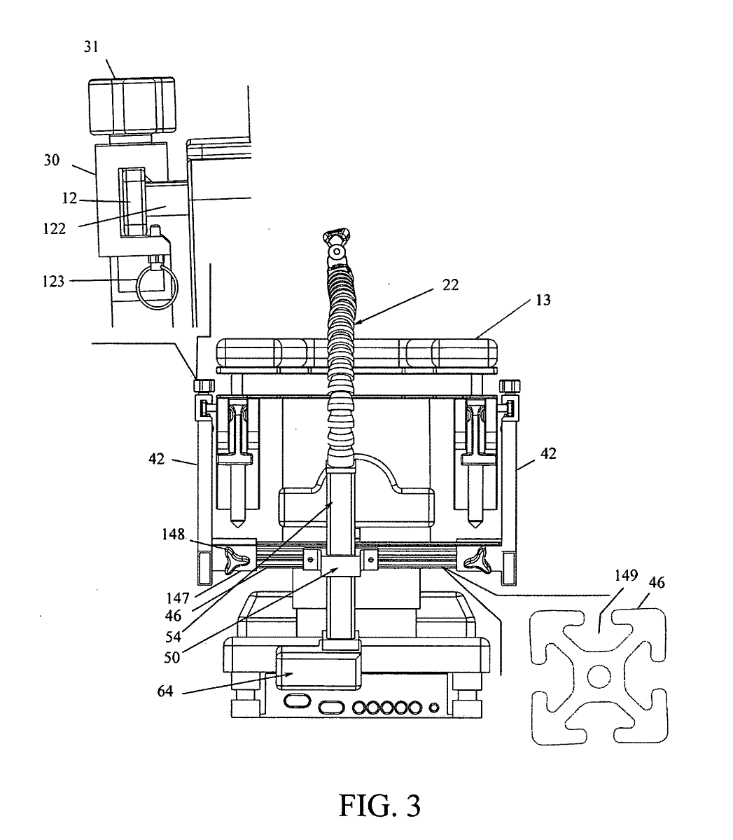

FIG. 3 is a composite front view of the stabilizer system 1 with enlarged insets. As seen at top left, the North American-standard side-rails 12 are elongate rails having a substantially rectangular cross-section mounted on opposing sides of the surgical table 13 on periodically-spaced stand-offs 122 which space them approximately 1″ outward from the table. Each quick-release bracket 30 is slidably positioned atop a side-rail 12, and comprises a C-shaped cross-section that embraces the side-rail 12, extending around and behind it. A pair of knobs 31 are threaded into the top of bracket 30 and extend there through for screw-insertion and manually clamping the bracket 30 to the side-rails 12. As a precaution, a safety pin 123 is inserted through a pre-drilled aperture in the bracket 30 behind the side-rail 12.

The downwardly protruding angle arms 42 are joined directly to the opposing collars 30 and (in FIG. 3) extend downward and forward around the foot of the table where they join the transverse support arm 46. The juncture may be accomplished with a bracket or receptacle 147 protruding inward from each angle arm 42. The transverse support arm 46 is seated in the opposing receptacles/brackets 147 and is carried there between. In this embodiment the transverse support arm 46 has a substantially square cross-section and receptacles 147 conform to it. Receptacles 147 may be equipped with set-screw knobs 148 journaled therein to allow quick-release mounting of the transverse support arm 46, although set screws will suffice. The set-screw knobs 148 can be manually loosened to facilitate removal of the transverse support arm 46. As seen at the bottom right inset of FIG. 3, the transverse support arm 46 has a substantially square (or rectangular) cross-section and is formed with lengthwise channels 149 along the length of each face. The transverse support arm 46 as shown in FIG. 3 can be formed from extruded aluminum. The collar 50 is a rectangular yoke with side-flanges attached to the forward channel of support arm 46. Collar 50 may be attached by set-screw knobs 148 similar to those of the receptacles 147 to allow loosening, by which the collar 50 may be laterally repositioned along the length of the support arm 46 or selectively locked in position.

The collar 50 carries the actuator 64 (by its protruding housing 54), and so the collar 50 also allows the actuator housing 54 to be extended upward or retracted downward therein, and clamped in place.

The actuator 64 can be a variety of different types. For example, the actuator 64 may be an electronic motor that selectively winds/unwinds a tensioning cable onto a pulley. Alternatively, the actuator 64 may be a linear actuator, or pneumatic cylinder (e.g., air cylinder), hydraulic cylinder, or any other suitable actuator capable of tightening/loosening a cable. The actuator 64 provides the motive force and incorporates control circuitry to selectively tighten/loosen the internal tensioning cable 240 which extends through actuator housing 54 to the flexible arm 22 mounted there atop. Preferably, actuator 64 includes an internal actuator control system constructed according to the type of actuation, and generically including one or more position sensors, a PLC controller, and hydraulic/pneumatic valves as necessary. By way of example, Duff-Norton™ sells a TracMaster™ line of linear actuators in a variety of stroke lengths, along with accessory control packages and digital position indicators that will suffice.

The flexible arm 22 comprises a plurality of links 230, optionally covered with a thin walled elastomeric sheath.

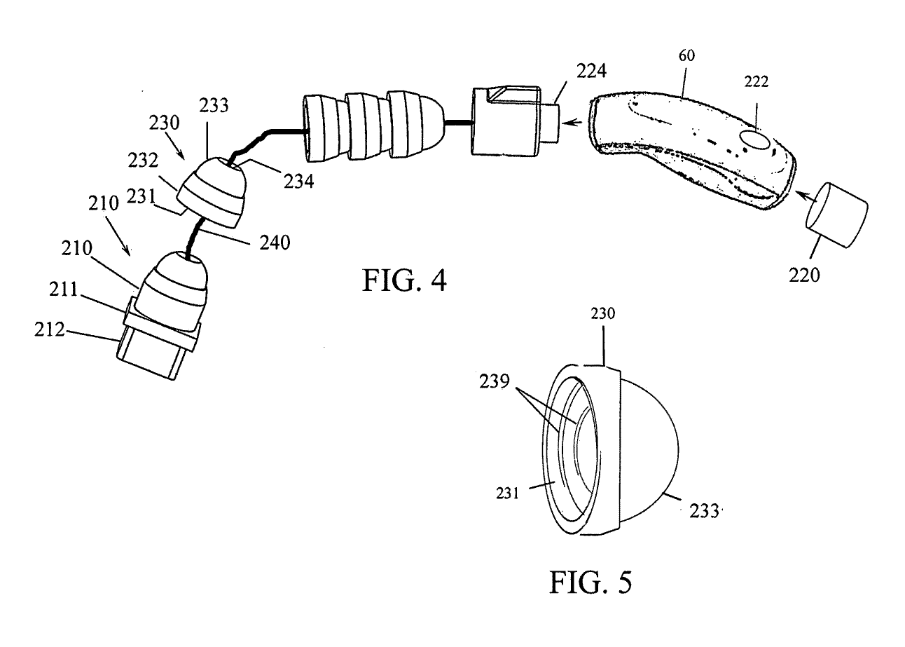

FIG. 4 illustrates the presently-preferred flexible arm configuration which generally includes an end adapter 210 at one end for insertion into the actuator housing 54, and a hand piece 60 at the other end for mounting the desired surgical tool that needs to be stabilized a plurality of ball-and-socket links 230 there between, and a tensioning cable 240 anchored in the hand piece 60 and running throughout the ball-and-socket links 230, end adapter 210 and actuator housing 54, and engaged to the actuator 64 for selectively tensioning or releasing ball-and-socket links 230. The ergonomic instrument-supporting hand piece 60 is mounted at the distal end of the flexible arm 22, and the hand piece 60 accepts a variety of surgical tool adapters 220 each of which is fitted to serve as a receptacle for insertion of a surgical tool (such as a vaginal probe 70 or cutting instrument, or camera, etc.). If desired (though not shown), ball-and-socket links 230 may be covered with a thin walled elastomeric sheath for aesthetics and possible sterilization improvement issues. The tensioning cable 240 may be any suitable twisted fiber cord or cable, and ⅛″ stainless steel wire rope is presently preferred. The cable 240 runs throughout the links 230 of the arm 22 and through the actuator housing 54 to the motor pulley (or actuator cylinder) in actuator 64, and in this way the actuator 64 can gradually pull (tension) the cable 240, which compresses the links 230 together to increase their collective rigidity, and ultimately lock them in position. There are a variety of alternative flexible arm configurations that may suffice for present purposes, the primary parameters being the ability to articulate mechanically in any direction, electrically or mechanically freeze a desired position along its entire length, and hold that position with maximum strength. The present configuration does this with between 14 and 22 ball-and-socket links 230 configured as shown. Several critical design features for these links 230 are described below which were developed to optimize the performance of the invention. First, it was found that the mating links 230 need to be constructed of a relatively high modulus material. The high modulus reduces local deformation in each link and keeps the distal end of the arm 22 from shifting when loaded in compression by the cable 240. Shifting of the distal end of the arm 22 when placed under load is not desirable since the arm is designed to hold and position precision surgical tools, cutting instruments, probes and/or cameras and could cause injury to the patient or complications for the user if movement occurs during the locking process. A range of materials can meet this requirement including some higher modulus plastics and a host of metal alloys. To enable the links to be injection molded (to reduce manufacturing costs), the current invention uses a 30% glass-filled thermoplastic material with a compressive modulus of elasticity of approximately 1,000,000 psi. Another important design feature is the geometry and size of the links 230. Generally, the larger the diameter of the link, the more holding strength the link and arm system can develop. The links 230 of the current invention each have a convex spherical face with a diameter preferably of about 1.5 inches, and within a range of from 0.5 to 4 inches. The geometry of the links is also important.

FIG. 5 is an enlarged illustration of an exemplary link 230 showing geometry. Each link is a ball-and-socket design, with a convex partial-spheric face or dome 233 at one end and a concave partial-spheric face or recess 231 at the other end. Each convex dome 233 on one link conforms to the concave recess 231 on the adjoining link, and so the ball-and-socket links 230 fit end-to-end. An important feature of this design is that the spherical diameter of the concave end 231 is purposefully designed to be slightly smaller than the mating convex end 233. This concentrates a portion of the reaction load along a circular line formed between the two mating spherical parts. This in turn increases the normal force along a circular line of contact, thereby increasing the friction loads and the moment carrying load capacity of the joint by maintaining an optimal friction loading geometry in each link. For the current invention, the concave end was sized to be 0.006 inches smaller in diameter than the convex part, although other combinations of material properties and sizes could also be used to achieve some measure of this feature. Another important design feature is related to the surface finish and material type, each of which can affect the friction coefficient between the mating parts. It is desirable to achieve a relatively high coefficient of friction between the mating links to increase the load holding capacity of the arm system. The current invention was designed to achieve a friction coefficient greater than 1.0. The presently-preferred surface finish comprises a coaxial series of circular ribs 239, or “hoop rings” in the convex surface 233 of each link 230.

The combination of the high modulus of elasticity material used for the links 230, high coefficient of friction between the mating links 230 (increased by controlled and uniform injection-molded surface finish, as well as the above-described optimum geometry of the links 230, has enabled the load carrying capacity of the above-described configuration to be substantially greater than prior art stabilizers. Specifically, once the arm 22 is locked the table mounted stabilizer system has a minimum load carrying capability of 2 ft-lbs torsionally, 25 pounds axially and 10 pounds laterally for rigid, reliable and secure support of any device.

Each link 230 is defined by an axial passage 234 for passing the cable 240, and a control wire to the button 222, and (optionally) for the delivery of negative air pressure (vacuum) for suction instruments. The end adapter 210 includes a square insert 212 backed by a flange 212 at one end for insertion into the square tubular actuator housing 54. Another ball-and-socket link identical to 230 protrudes integrally from the other side of flange 212.

The hand piece 60 allows 360 degree rotation of the instrument wielded thereby. The ergonomic hand piece 60 is a contoured member having the arm control button 222 mounted there atop. The throw of actuator 64 is controlled by the button 222 on the instrument-supporting hand piece 60. The hand piece 60 includes an open-ended receptacle for insertion of an adapter 220 that will interface with and support a variety of desired surgical tools. The adapter 220 preferably allows snap-fit insertion of a vaginal probe 70 or other device.

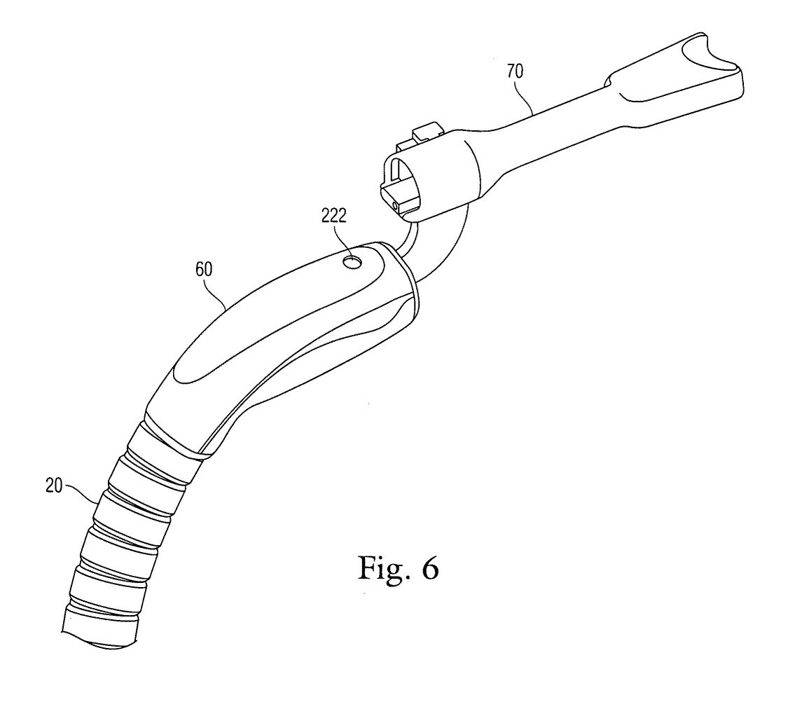

FIG. 6 is an enlarged side-view of the flexible arm 20 supporting the hand piece 60, which is in turn carrying a vaginal probe 70. The probe-adapter inserts 220 may assume various internal configurations as needed for snap-fit insertion and secure holding of any of a variety of surgical tools (such as vaginal probe 70, or surgical tools, cutting instruments, cameras or the like). This effectively makes the table mounted stabilizer system universally capable of holding most any instrument, universally capable of manipulating that instrument into any desired position and angle, and then locking it in fixed position, all with a single hand (or optional voice activated control) by a surgeon or surgical assistant.

One skilled in the art should now understand that the foregoing allows full adjustability of the location of the desired instrument up and down along a vertical axis, forward and back, i.e, toward or away from the patient, and rotationally so that the desired instrument can be angled up or down and side to side. Once locked the table mounted stabilizer system has a minimum load carrying capability of 2 ft-lbs torsionally, 25 pounds axially and 10 pounds laterally for rigid, reliable and secure support of any device. Moreover, the hand piece allows single-handed release control of all degrees of freedom.

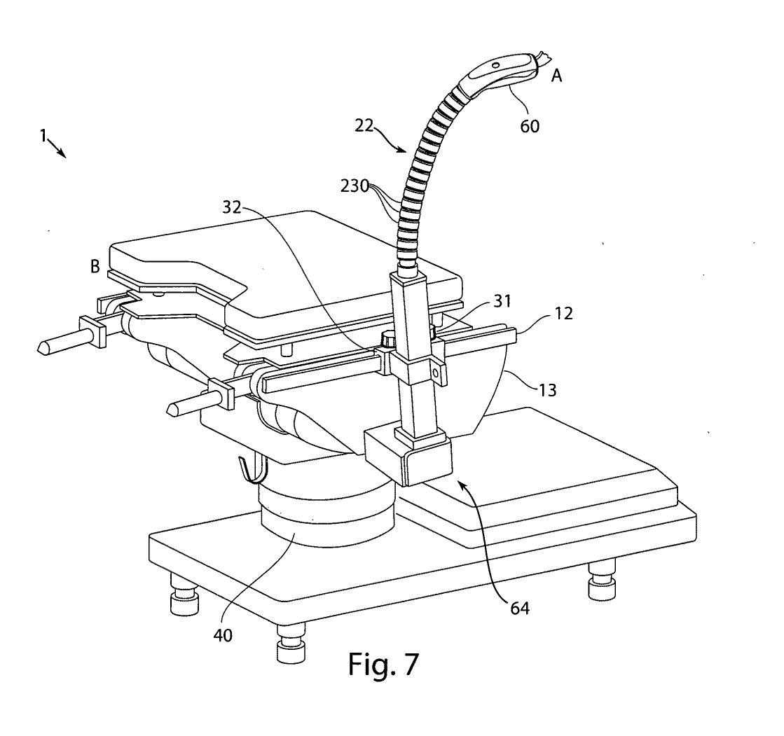

FIG. 7 is a perspective view of an alternative scheme of the stabilizer system 1 in which the collar 50 and associated components are affixed directly to one of the existing horizontal side rails 12, without the use of the U-shaped frame 40. All other components are the same and like reference numbers are used. While this embodiment compromises a degree of articulation it still provides the advantages of the flexible arm in a simple configuration.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A surgical instrument stabilizer system for holding and repositioning a variety of medical instruments relative to a surgical table during a procedure, said surgical table including a patient platform with at least one side rail attached alongside said patient platform, the surgical instrument stabilizer system comprising:
a rigid arm assembly attached at one end to said at least one side rail by a first locking bracket for slidable translation there along;
a transverse beam attached to another end of said rigid arm assembly;
an articulating boom supported by said transverse beam, said articulating boom further comprising,
an actuator,
a multi-directional flexible arm attached at one end to said actuator, and
a cable connected to said actuator for extension/retraction thereby, said cable extending through said flexible arm for compressing said flexible arm upon retraction and release upon extension, thereby selectively fixing said flexible arm is a desired configuration; and
an instrument-supporting member attached distally at another end of said flexible arm for insertion of a medical instrument therein;
whereby a medical instrument inserted in said instrument-supporting member can be repositioned and locked in a desired position and orientation single-handedly.

2. The surgical instrument stabilizer system according to claim 1, wherein said actuator comprises a motor.

3. The surgical instrument stabilizer system according to claim 1, wherein said actuator comprises a pressurized cylinder.

4. The surgical instrument stabilizer system according to claim 1, wherein said instrument-supporting member comprises a hand piece having a receptacle.

5. The surgical instrument stabilizer system according to claim 4, further comprising an adaptor inserted in the receptacle of said hand piece for coupling a medical instrument therein.

6. The surgical instrument stabilizer system according to claim 4, wherein said hand piece comprises a control switch in communication with said actuator for selective extension/retraction of said cable.

7. The surgical instrument stabilizer system according to claim 1, wherein said flexible arm comprises a plurality of ball-and-socket links.

8. The surgical instrument stabilizer system according to claim 7, wherein each of said ball-and-socket links comprises a glass-filled thermoplastic member having a compressive modulus of elasticity of at least 1,000,000 psi.

9. The surgical instrument stabilizer system according to claim 7, wherein each of said ball-and-socket links comprises a convex partial-spheric face with a diameter in a range of from ½ to 4 inches

10. The surgical instrument stabilizer system according to claim 7, wherein each of said ball-and-socket links comprises a convex partial-spheric face on one side and a concave partial-spheric face on another side, a spherical diameter of the concave face being smaller than a spherical diameter of the convex face to increase load carrying capacity between adjacent ball and socket links.

11. The surgical instrument stabilizer system according to claim 1, whereby a medical instrument inserted in said instrument-supporting member can be repositioned and locked in any position and orientation along any axis within a three-dimensional space above said platform.

12. A surgical instrument stabilizer system for holding and repositioning a medical instrument relative to a surgical platform during a procedure, comprising:
a pair of side rails attached on opposing sides of said surgical platform;
a pair of rigid arms movably attached to corresponding side rails by a pair of first locking brackets for slidable translation there along;
a beam attached to between said pair of rigid arms and having a second locking bracket movably mounted there along for slidable translation along said beam;
a flexible arm assembly attached at one end to said second locking bracket, said flexible arm assembly further comprising a multi-directional articulating arm and a locking mechanism for locking said arm in a desired position; and
an instrument-supporting member attached at another end of said flexible arm;
whereby a medical instrument supported in said instrument-supporting member can be repositioned quickly and easily, and locked in any desired position and orientation within a three-dimensional space above said platform.

13. The surgical instrument stabilizer system according to claim 12, wherein said pair of rigid arms extend parallely downward at an angle from said first locking brackets.

14. The surgical instrument stabilizer system according to claim 13, wherein said first locking brackets comprise slide-clamps for selective clamping to a side rail.

15. The surgical instrument stabilizer system according to claim 14, wherein said transverse beam comprises an extruded aluminum beam formed with a track there along.

16. The surgical instrument stabilizer system according to claim 15, wherein said second locking bracket comprises a screw-clamp channeled within the track of said transverse beam.

17. The surgical instrument stabilizer system according to claim 16, wherein said second locking bracket comprises a collar attached to said screw-clamp for extensible mounting of said flexible arm assembly.

18. The surgical instrument stabilizer system according to claim 12, wherein said flexible arm assembly further comprises a plurality of ball-and-socket links.

19. The surgical instrument stabilizer system according to claim 18, wherein each of said ball-and-socket links comprises a convex partial-spheric face with a diameter in a range of from ½ to 4 inches

20. The surgical instrument stabilizer system according to claim 18, wherein each of said ball-and-socket links comprises a convex partial-spheric face on one side and a concave partial-spheric face on another side, a spherical diameter of the concave face being smaller than a spherical diameter of the convex face to increase load carrying capacity between adjacent ball and socket links.

21. The surgical instrument stabilizer system according to claim 18, wherein said flexible arm further comprises a tensioning cable passing through the holes of said ball-and-socket links.

22. The surgical instrument stabilizer system according to claim 12, further comprising an actuator for imparting progressively more or less rigidity to said flexible arm.

23. The surgical instrument stabilizer system according to claim 22, wherein said instrument supporting member comprises a hand piece with a control switch in communication with said actuator for controlling the rigidity imparted to said flexible arm by said actuator.

24. The surgical instrument stabilizer system according to claim 22, further comprising a voice recognition controller for controlling said actuator.

25. The surgical instrument stabilizer system according to claim 18, wherein said flexible arm further comprises said ball-and-socket links each formed with a center passage for delivering vacuum to surgical tools.

26. A stabilizer system for holding and repositioning of an instrument relative to a platform, comprising:
a supporting frame attached to said platform;
a flexible arm assembly attached to said supporting frame, said flexible arm assembly further comprising,
an actuator,
a multi-directional flexible arm attached to said actuator, said multi-directional flexible arm further comprising a plurality of ball-and-socket links, and
a cable connected to said actuator for extension/retraction thereby, said cable extending through the plurality of ball-and-socket links of said multi-directional flexible arm for selectively compressing the links of said flexible arm and thereby fixing said flexible arm in a desired configuration; and
an instrument-supporting member attached distally at another end of said flexible arm for insertion of an instrument therein;
whereby an instrument inserted in said instrument-supporting member can be repositioned and locked in a desired position and orientation.

27. The stabilizer system according to claim 24, wherein each of said ball-and-socket links of said multi-directional flexible arm comprises a convex ball at one end, a concave sphere at another end, and a though hole running from end-to-end.

28. The stabilizer system according to claim 25, wherein said plurality of ball-and-socket links of said multi-directional flexible arm each have a modulus of elasticity of at least 500,000 psi.

29. The stabilizer system according to claim 25, wherein said plurality of ball-and-socket links of said multi-directional flexible arm are formed with a bearing surface finish to increase their coefficient of friction.

30. The stabilizer system according to claim 27, wherein the plurality of ball-and-socket links may be fully compressed to lock said flexible arm in a fixed configuration providing a minimum load carrying capability of approximately 2 ft-lbs torsionally, approximately 25 pounds axially, and approximately 10 pounds laterally for rigid, reliable and secure support of any device.

31. The stabilizer system according to claim 24, wherein said frame comprises a pair of arms extending downward at an angle from opposing sides of said platform.

32. The stabilizer system according to claim 29, wherein said pair of arms are attached to said platform by slide-clamps for selective clamping along said platform.

33. The stabilizer system according to claim 29, wherein said frame comprises a transverse beam supported between said pair of arms, and said beam includes a defined track there along.

34. The stabilizer system according to claim 31, further comprising a second slidable locking bracket including a screw-clamp channeled within the track of said transverse beam.

35. The stabilizer system according to claim 32, wherein said second slidable locking bracket includes a clamping collar for extensible mounting of said flexible arm assembly.

36. A surgical instrument stabilizer system for holding and single-handed or voice activated repositioning of an instrument relative to a surgical platform during a procedure, comprising:
an articulating arm assembly having a plurality of discrete links connected together for multi-directional articulation, and an instrument-supporting hand piece attached at one end of said plurality of discrete links;
a mechanism for locking said articulating arm from a flexible to a desired inflexible position;
whereby a supported instrument attached to said hand piece can be repositioned quickly and easily by single-handed or voice activated manipulation, and locked in any position and orientation along any axis within a three-dimensional space above said platform.

37. The surgical instrument stabilizer system according to claim 34, wherein said articulating arm assembly further comprises a plurality of ball-and-socket links.

38. The surgical instrument stabilizer system according to claim 37, wherein each of said ball-and-socket links comprises a convex partial-spheric face with a diameter in a range of from 1/2 to 4 inches

39. The surgical instrument stabilizer system according to claim 37, wherein each of said ball-and-socket links comprises a convex partial-spheric face on one side and a concave partial-spheric face on another side, a spherical diameter of the concave face being smaller than a spherical diameter of the convex face to increase load carrying capacity between adjacent ball and socket links.

40. The surgical instrument stabilizer system according to claim 36, wherein said mechanism for adjustably locking said articulating arm further comprises a tensioning cable attached at one end to an actuator and at another end to said articulating arm for imparting progressively more or less rigidity to said articulating arm by controlling the amount of tension in the tensioning cable.

41. The surgical instrument stabilizer system according to claim 36, further comprising a control switch on said instrument-supporting hand piece for controlling said actuator by one of electrical, optical or wireless communication.

42. A surgical instrument stabilizer system for holding and repositioning a variety of medical instruments relative to a surgical table during a procedure, said surgical table including a patient platform with at least one side rail attached alongside said patient platform, the surgical instrument stabilizer system comprising:
an articulating boom supported by said to at least one side rail by a first locking bracket for slidable translation there along, said articulating boom further comprising,
an actuator,
a multi-directional flexible arm attached at one end to said actuator, and
a cable connected to said actuator for extension/retraction thereby, said cable extending through said flexible arm for compressing said flexible arm upon retraction and release upon extension, thereby selectively fixing said flexible arm is a desired configuration; and
an instrument-supporting member attached distally at another end of said flexible arm for insertion of a medical instrument therein;
whereby a medical instrument inserted in said instrument-supporting member can be repositioned and locked in a desired position and orientation single-handedly.
